(19) United States
(12) Patent Application Publication
(10) Pub. No.: US 2008/0014223 A1
(43) Pub. Date: Jan. 17, 2008

(54) Syrup Composition Comprising Dexibupropen as an Active Ingredient and Method for the Preparation Thereof

(75) Inventors: Jong Soo Woo, Suwon-si (KR); Hong Gi Yi, Suwon-si (KR); Ju Nam Jin, Pohang-si (KR)

Correspondence Address: SUGHRUE MION, PLLC, 2100 PENNSYLVANIA AVENUE, N.W., SUITE 800, WASHINGTON, DC, 20037, US

(73) Assignee: Hammi Pharm. Co., Ltd., Hwaseong-gun (KR)

(21) Appl. No.: 11/813,315

(22) PCT Filed: Jan. 3, 2006
(86) PCT No.: PCT/KR06/00016

§ 371 Date: Jul. 3, 2007

(30) Foreign Application Priority Data
Jan. 3, 2005 (KR) 10-2005-0000222

(51) Int. Cl. A61K 9/08; A61K 31/19; A61P 29/00

(52) U.S. Cl. 424/400; 514/570

(57) ABSTRACT

A dexibupropen syrup composition comprising dexibupropen ((S)-ibupropen) having an average particle size ranging from 10 to 300 μm as an active ingredient, which does not contain glycerine and has a viscosity ranging from 500 to 3,000 cps and pH ranging from 3.0 to 6.0, shows improved safety, uniformity of efficacy, and taste.

### FIELD OF THE INVENTION

The present invention relates to a glycerin-free dexibupropen syrup composition having enhanced stability which comprises dexibupropen ((S)-ibupropen) having an average particle size ranging from 10 to 300 μm as an active ingredient, said composition having a viscosity ranging from 500 to 3,000 cps and pH ranging from 3.0 to 6.0, and a method for the preparation thereof.

### BACKGROUND OF THE INVENTION

Ibupropen is a representative propionic acid-based non steroidal anti-inflammatory drug, which acts as a powerful antiphlogistic and analgesic by inhibiting the cyclooxygenase activity in the biosynthesis of prostaglandin, and thus, it is widely used for treating diseases such as rheumatoid arthritis, arthralgia, tendonitis, gout and ankylosing spondylitis, as well as for soothing the pain and inflammation after a surgical operation.

Ibupropen exists in the form of a racemate consisting of equal amounts of two optical isomers, (S)— and (R)—, but the pharmaceutically active isomer is the (S)-ibupropen (dexibupropen). Therefore, a drug comprising only the pharmaceutical active (S)-ibupropen exhibits the expected pharmaceutical effect at a smaller dosage, and excludes possible side effects caused by the pharmaceutically inactive (R)-ibupropen.

Up to now, various syrups comprising dexibupropen have been prepared. For example, Korean patent publication 2004-51826 discloses a method for the preparation of a dexibupropen syrup by solubilizing dexibupropen using a plasticizer composed of concentrated glycerin and polyoxyl 40-hardened castor oil, and shielding the stinging taste of the drug by adding a flavoring agent. However, there has been a continual need to develop an improved dexibupropen syrup for administration to children, which has better taste and good storage stability without phase separation or precipitate formation.

### SUMMARY OF THE INVENTION

Accordingly, it is an object of the present invention to provide a dexibupropen syrup composition having improved safety, stability, consistency of the pharmaceutical effect, texture and taste, and a method for the preparation thereof.

In accordance with one aspect of the present invention, there is provided a glycerin-free dexibupropen syrup composition comprising dexibupropen ((S)-ibupropen) having an average particle size ranging from 10 to 300 μm as an active ingredient, said composition having a viscosity ranging from 500 to 3,000 cps and pH ranging from 3.0 to 6.0.

### DETAILED DESCRIPTION OF THE INVENTION

The present invention provides a syrup composition comprising a specific form of dexibupropen as an active ingredient and optionally an excipient such as a viscosity controlling agent, a sweetener, a suspending agent, an emulsifier, a pH controlling agent, a preservative, a colorant, a flavoring agent and a solvent.

(1) Active Ingredient

The active ingredient of the inventive composition, dexibupropen, is employed in an amount ranging from 0.01 to 10.0 w/v %, preferably 0.7 to 5.0 w/v % based on the total volume of the syrup composition, in the form of particles having an average particle size in the range from 10 to 300 μm to prevent the precipitation of dexibupropen and minimize the sandy texture of the particles in the mouth.

(2) Viscosity Controlling Agent

In the present invention, a viscosity controlling agent may be used to control the viscosity of the composition in the range from 500 to 3,000 cps, and it is selected from the group consisting of agar, sodium alginate, povidone, polyethylene glycol, hydroxyethylene cellulose, D-sorbitol solution and a mixture thereof. The viscosity controlling agent prevents layer separation of the dexibupropen syrup composition, and provides proper fluidity for oral administration to children. The agent may be employed in an amount ranging from 0.01 to 40.0 w/v %, preferably 0.1 to 30.0 w/v % based on the total volume of the syrup composition.

(3) Sweetener

In the present invention, a sweetener may be used as an optional component and it is selected from the group consisting of sugar, high fructose, stevioside, dipotassium glycirhizinate and a mixture thereof suitable for administration to children. The sweetener may be employed in an amount ranging from 0.1 to 80.0 w/v %, preferably 0.1 to 70.0 w/v % based on the total volume of the syrup composition.

(4) Suspending Agent

In the present invention, a suspending agent may be used to suspend the above mentioned dexibupropen particles uniformly in the syrup composition, and it is selected from the group consisting of caoline, xanthan gum, agar and a mixture thereof. The suspending agent may be employed in an amount ranging from 0.01 to 10.0 w/v %, preferably 0.2 to 5.0 w/v % based on the total volume of the syrup composition.

(5) Emulsifier

In the present invention, an emulsifier may be used to emulsify a suspension of the active ingredient, and it can be any one of polysorbate compounds or a mixture thereof. The emulsifier may be employed in an amount ranging from 0.01 to 5.0 w/v %, preferably 0.05 to 3.0 w/v % based on the total volume of the syrup composition.

(6) pH Controlling Agent

In the present invention, a pH controlling agent may be used to eliminate the bitter and puckery taste of the dexibupropen syrup composition by controlling the composition's pH in the range of 3 to 6, and it can be selected from the group consisting of citric acid, sodium citrate and a mixture thereof. The pH controlling agent may be employed in an amount ranging from 0.01 to 5.0 w/v %, preferably 0.1 to 1.0 w/v % based on the total volume of the syrup composition.

Further, the syrup composition of the present invention may further comprise a pharmaceutically acceptable additive such as a preservative selected from the group consisting of methyl parahydroxybenzoate, propyl parahydroxybenzoate and sodium benzoate; a colorant; a flavoring agent; or a solvent.

The inventive pharmaceutical composition comprising dexibupropen as an active ingredient can be prepared by a method comprising the steps of:

(a) dispersing the viscosity controlling agent and a portion of a predetermined amount of the sweetener in distilled water, stirring the resulting mixture at 80 to 90° C. for 1 to 6 hours to obtain a homogeneous solution, adding thereto the preservative, and stirring the resulting solution;

(b) dissolving the remaining portion of the predetermined amounts of the sweetener in the solution obtained in (a) clearly while maintaining the solution at 75 to 85° C., adding thereto the viscosity controlling agent and the pH controlling agent, and dissolving the resulting solution completely;

(c) cooling the solution obtained in (b) to 25 to 29° C. by adding cool water;

(d) adding a dexibupropen dispersion obtained by dispersing the active ingredient and the suspending agent to an aqueous emulsion containing the colorant and the emulsifier, and stirring the resulting mixture for 0.5 to 6 hours to obtain a suspension; and

(e) dispersing the solution obtained in (c) in the suspension obtained in (d), adding the flavoring agent, and mixing the resulting mixture uniformly.

Each component and amount thereof used the preparation method is as stated above.

The inventive syrup composition comprising dexibupropen as the active ingredient can be administered orally in the representative amount listed in Table 1 in a single dose or in divided 3 to 4 doses.

TABLE 1

| Age | Single dosage (mg) |
|---|---|
| 11 to 14 | 120 to 150 |
| 7 to 10 | 90 to 120 |
| 3 to 6 | 60 to 90 |
| 1 to 2 | 30 to 60 |

The inventive composition, which uses dexibupropen corresponding to the (s)-isomer, not ibupropen consisting of (R)— and (S)-isomers, can be administered at a reduced dosage without side effects, and has improved safety, stability, consistency of the pharmaceutical effect, texture and taste. Therefore, it can be broadly used for treating diseases such as rheumatoid arthritis, arthralgia, tendonitis, gout and ankylosing spondylitis, as well as for soothing the pain and inflammation after a surgical operation.

The following Examples are intended to further illustrate the present invention without limiting its scope.

### PREPARATION EXAMPLE

### General Preparation Procedure For a Syrup Composition

1) Distilled water was poured into a preparation tank, and a portion of a predetermined amount of sugar (corresponding to a sugar content of 27.5 w/v % in the final composition) and agar were dispersed therein, and the dispersion was stirred at 85 to 90° C. for about 3 hours to obtain a clear solution;

2) a preservative mixture composed of methyl parahydroxybenzoate, propyl parahydroxybenzoate and sodium benzoate was added to the solution obtained in 1), and dissolved by stirring;

3) the remaining portion of the predetermined amount of sugar was added to the solution obtained in 2) which was maintained at 75 to 85° C., and stirred until the mixture became clear;

4) predetermined amounts of high fructose (Doosan corn products Korea), D-sorbitol solution (70%, Roqquette), citric acid and sodium citrate were added to the solution obtained in 3) maintained at 70° C.;

5) tar (Bolak) and polysorbate 80 (Uniquma LAB) were added to distilled water, and emulsified by mixing;

6) a dispersion mixture of dexibupropen and caoline was suspended in the solution obtained in 5) for about 3 hours;

7) the solution obtained in 4) was cooled to 25 to 29° C. by adding thereto a small amount (about 1.0 w/v %) of cool water, which was added to the suspension obtained in 6), followed by adding thereto lime essence (Hanmi Perfumery & Chemical Co., Ltd); and

8) distilled water was added to the mixture obtained in 7) to adjust the final total volume and mixed uniformly.

### EXAMPLE 1 AND COMPARATIVE EXAMPLES 1 TO 3

A dexibupropen syrup composition having the components listed in Table 2 was prepared in accordance with the procedure of the Preparation Example (Example 1). This composition did not contain stability-reducing glycerin.

In addition, three comparative dexibupropen syrup compositions having the components listed in Table 2 were prepared by repeating the procedure of Example 1 except for adding 5.0, 10.0 and 20.0 g of glycerin as a viscosity controlling agent, respectively in step 4) of the Preparation Example together with high fructose and D-sorbitol solution (Comparative Examples 1 to 3).

TABLE 2
Dexibupropen syrup composition (100 ml)

| | | | Comparative | Comparative | Comparative |
|---|---|---|---|---|---|
| Ingredient | | Example 1 | Example 1 | Example 2 | Example 3 |
| pH | | 3.7 | 3.8 | 3.7 | 3.7 |
| Active | Dexibupropen | 1.2 g | 1.2 g | 1.2 g | 1.2 g |
| ingredient | (50 μm) | | | | |
| (average | | | | | |
| particle size) | | | | | |
| Sweetener | Sugar | 45.0 g | 45.0 g | 45.0 g | 45.0 g |
| | High fructose | 30.0 g | 30.0 g | 30.0 g | 30.0 g |
| Viscosity | Agar | 0.3 g | 0.3 g | 0.3 g | 0.3 g |
| controlling | D-sorbitol solution | 21.0 g | 21.0 g | 21.0 g | 21.0 g |
| agent | Glycerin | — | 5.0 g | 10.0 g | 20.0 g |
| Suspending | Light caoline | 1.1 g | 1.1 g | 1.1 g | 1.1 g |
| agent | | | | | |
| Emulsifier | Polysorbate 80 | 0.12 g | 0.12 g | 0.12 g | 0.12 g |
| Preservative | Methyl para | 0.03 g | 0.03 g | 0.03 g | 0.03 g |
| | hydroxybenzoate | | | | |
| | Propyl para | 0.02 g | 0.02 g | 0.02 g | 0.02 g |
| | hydroxybenzoate | | | | |
| | Sodium benzoate | 0.05 g | 0.05 g | 0.05 g | 0.05 g |
| Colorant | Tar | 0.01 g | 0.01 g | 0.01 g | 0.01 g |
| | (KFDA Notification | | | | |
| | NO. 2000-66) | | | | |
| Flavoring | Lime essence | 0.09 g | 0.09 g | 0.09 g | 0.09 g |
| agent | | | | | |
| pH | Citric acid | 0.24 g | 0.24 g | 0.24 g | 0.24 g |
| controlling | Sodium citrate | 0.10 g | 0.10 g | 0.10 g | 0.10 g |
| agent | | | | | |
| solvent | Distilled water | Adjust to | Adjust to | Adjust to | Adjust to |
| | | 100 ml | 100 ml | 100 ml | 100 ml |

### TEST EXAMPLE 1

### The Stability of a Dexibupropen Syrup Composition and its Glycerin Content

To compare the stabilities of the dexibupropen syrup compositions prepared in Example 1 and Comparative Examples 1 to 3, the compositions were stored under an accelerated aging condition (40° C. and relative humidity 75%) in accordance with KFDA (Korea Food and Drug Administration) Notification No. 2000-7, and time-dependent amounts of degradation products of dexibupropen were analyzed under the following conditions:

[Analysis Method: Liquid Chromatography]

Column—Stainless column packed with octadecyl silylated silica gel (150 mm×4.6 mm, 5 μm, Inertsil ODS-2, GL Science Inc, Japan),

Mobile phase—acetonitrile:water:phosphoric acid=600:400:0.5 (v/v/v),

Injection volume—10 μl,

Flow rate—1.2 ml/min, and

Detection—UV spectrophotometer (wavelength: 214 nm, L-7400, Hitachi, Japan)

KFDA regulation states that the amount of 2-(4-isobutylphenyl)-propionic acid methyl ester produced as a disintegrant of dexibupropen should be 0.3 weight % and less, its relative peak retention time under the above LC condition being 2.65 min, and that the total amount of unknown disintegrants should be 0.3 weight % and less.

The results are shown in Table 3.

TABLE 3
Production rate (%) of an unknown disintegrant (relative peak retention
time: 0.64 min)

| | | Comparative | Comparative | Comparative |
|---|---|---|---|---|
| | Example 1 | Example 1 | Example 2 | Example 3 |
| Glycerin | 0 | 5 | 10 | 20 |
| (g/100 ml) | | | | |
| Initial | 0.00 | 0.00 | 0.00 | 0.00 |
| After 3 months | 0.00 | 0.21 | 0.29 | 0.37 |
| After 6 months | 0.00 | 0.45 | 0.54 | 0.68 |

As can be seen in Table 3, the dexibupropen syrup composition of Example 1 containing no glycerin did not produce any unknown disintegrant, while the compositions of Comparative Examples 1 to 3 containing varying amounts of glycerin produced an unknown disintegrant in a time and glycerin content-dependent manner. Therefore, the inventive dexibupropen syrup composition is much more stable and safe than those conventional dexibupropen compositions containing glycerin.

### EXAMPLES 2 TO 5 AND COMPARATIVE EXAMPLES 4 AND 5

Additional dexibupropen syrup compositions were prepared by repeating the procedure of Example 1 except for using dexibupropen particles having average particle sizes of 10, 50, 100 and 300 μm, respectively (Examples 2 to 5).

In addition, two comparative dexibupropen syrup compositions were prepared by repeating the procedure of Example 1 except for using dexibupropen particles having average particle sizes 400 and 500 μm, respectively (Comparative Examples 4 and 5).

### TEST EXAMPLE 2

### The Effect of the Average Dexibupropen Particle Size of a Dexibupropen Syrup Composition on the Stability

The dexibupropen syrup compositions prepared in Examples 2 to 5 and Comparative Examples 4 and 5 were each centrifuged (2,000 rpm, 20 mins), and observed for the presence of any precipitant. The results are shown in Table 4.

Further, the dexibupropen syrup compositions prepared in Examples 2 to 5 and Comparative Examples 4 and 5 were each orally administered to a group of randomly selected 10 men and 10 women, and the each member of the group was asked whether the subject felt roughness in the mouth. The results are shown in Table 4 according to the following criteria:

−: feels no roughness in the mouth possibly caused by any particle,

+: feels slight roughness in the mouth caused by particles, but it is tolerable,

++: feels some roughness in the mouth, and

+++: feels severe roughness in the mouth.

TABLE 4

| | | | | | Comparative | Comparative |
|---|---|---|---|---|---|---|
| | Example 2 | Example 3 | Example 4 | Example 5 | Example 4 | Example 5 |
| Average | 10 | 50 | 100 | 300 | 400 | 500 |
| particle size | | | | | | |
| (μm) | | | | | | |
| Precipitant | X | X | X | ◯ | ◯ | ◯ |
| (thickness) | | | | (0.1 cm) | (0.4 cm) | (0.7 cm) |
| Roughness | − | − | + | + | +++ | +++ |
| after | | | | | | |
| administration | | | | | | |

As can be seen in Table 4, the dexibupropen syrup compositions having an average particle size over 400 μm produced large amounts of precipitants, which cause the problems of the homogeneity and roughness feeling in the mouth of a recipient patient administrated with dexibupropen composition.

### COMPARATIVE EXAMPLES 6 TO 8

Three dexibupropen syrup compositions were prepared by repeating the procedure of Example 1 except for using 0.15, 0.45 and 0.60 g of agar as a viscosity controlling agent, respectively.

### TEST EXAMPLE 3

### The Effects of the Viscosity of Dexibupropen Syrup Composition on the Stability and Fluidity

The viscosities of dexibupropen syrup compositions prepared in Example 1 and Comparative Examples 6 to 8 were each measured with a viscometer (Brookfield viscometer, USA/LV model, No. 2 spindle, 12 rpm). Also, the susceptibility of each composition to layer separation was examined by centrifuging the composition (2,000 rpm, 20 mins, MF 550, Hanil Science Industrial), and measuring the amount of the supernatant. The relative fluidity was compared by measuring the time a 1 ml sample composition, placed on a 45° slope at a spot 10 cm apart from the bottom of the slope, took to reach the bottom. The results are shown in Table 5.

TABLE 5
The effects of the viscosity of a dexibupropen syrup composition on
the stability and fluidity

| | | Comparative | Comparative | Comparative |
|---|---|---|---|---|
| | Example 1 | Example 6 | Example 7 | Example 8 |
| Agar | 0.30 | 0.15 | 0.45 | 0.60 |
| (g/100 ml) | | | | |
| Viscosity (cps) | 1540 | 370 | 3510 | 4250 |
| layer | 0.1 | ≧0.4 | <0.1 | 0.0 |
| separation (ml) | | | | |
| fluidity | Good | Good | Poor | Not fluid |

As can be seen in Table 5, an excessively low viscosity of the composition causes layer separation, while an unnecessarily high viscosity causes difficulties in administrating the syrup composition to children.

### EXAMPLE 6

A dexibupropen syrup composition was prepared by repeating the procedure of Example 1 except for adding 0.03w/v % of citric acid to adjust pH to 3.0.

### EXAMPLES 7 TO 9 AND COMPARATIVE EXAMPLES 9 TO 11

Three dexibupropen syrup compositions were prepared by repeating the procedure of Example 1 except for adding 0.1 N NaOH to adjust pH to 4.0, 5.0 and 6.0, respectively (Examples 7 to 9).

In addition, three comparative dexibupropen syrup compositions were prepared by repeating the procedure of Example 1 except for adding 0.1 N NaOH to adjust pH to 7.0, 8.0 and 9.0, respectively (Comparative Examples 9 to 11).

### TEST EXAMPLE 4

### The Effect of pH of a Dexibupropen Syrup Composition on the Taste

The dexibupropen syrup compositions prepared in Examples 6 to 9 and Comparative Examples 9 to 11 were each administered to a group of 10 men and 10 women (20 to 30 years old), and the taste was evaluated under the following criteria. The results are shown in Table 6:

A: sweet and agreeable,

B: sweet but puckery after taste,

C: a little bitter or puckery after taste, and

D: very bitter or strongly puckery.

TABLE 6
The effect of pH of a dexibupropen syrup composition on the taste

| | | | | | Comparative | Comparative | Comparative |
|---|---|---|---|---|---|---|---|
| | Example 6 | Example 7 | Example 8 | Example 9 | Example 9 | Example 10 | Example 11 |
| pH | 3.0 | 4.0 | 5.0 | 6.0 | 7.0 | 8.0 | 9.0 |
| A | 16 | 18 | 15 | 10 | 3 | 0 | 0 |
| (persons) | | | | | | | |
| B | 3 | 1 | 3 | 7 | 6 | 3 | 4 |
| (persons) | | | | | | | |
| C | 1 | 1 | 2 | 2 | 8 | 10 | 2 |
| (persons) | | | | | | | |
| D | 0 | 0 | 0 | 1 | 3 | 7 | 14 |
| (persons) | | | | | | | |

As can be seen in Table 6, over 85% of the persons evaluated that the dexibupropen syrup compositions having pH ranging from 3 to 6 were easy to take (A and B), while the compositions having pH over 7.0 tasted bitter or puckery.

### EXAMPLES 10 TO 21

Dexibupropen syrup compositions having the components listed in Tables 7 to 9 were prepared by repeating the procedure of Example 1.

TABLE 7

| | | Exam- | Example | Example | Exam- |
|---|---|---|---|---|---|
| Ingredient (g) | | ple 10 | 11 | 12 | ple 13 |
| Sweetener | Sugar | 40.0 | 40.0 | 40.0 | 43.0 |
| | High fructose | 30.0 | 30.0 | 30.0 | 30.0 |
| Viscosity | Agar | 0.36 | 0.39 | 0.3 | 0.2 |
| controlling | Hydroxyethylene | — | — | 0.1 | 0.2 |
| agent | cellulose | | | | |
| | D-sorbitol | 26.0 | 23.0 | 26.0 | 23.0 |
| | solution | | | | |

TABLE 8

| | | Example | Example | Example | Example |
|---|---|---|---|---|---|
| Ingredient (g) | | 14 | 15 | 16 | 17 |
| Sweetener | Sugar | 40.0 | 43.0 | 50.0 | 45.0 |
| | High | 40.0 | 30.0 | 30.0 | 30.0 |
| | fructose | | | | |
| Viscosity | Agar | 0.3 | 0.3 | 0.4 | — |
| controlling | Povidone | — | — | — | 0.4 |
| agent | K-30 | | | | |
| | D-sorbitol | 21.0 | 23.0 | 10.0 | 21.0 |
| | solution | | | | |

TABLE 9

| | | Exam- | Example | Example | Exam- |
|---|---|---|---|---|---|
| Ingredient (g) | | ple 18 | 19 | 20 | ple 21 |
| Sweetener | Sugar | 50.0 | 40.0 | 40.0 | 30.0 |
| | High fructose | 10.0 | 20.0 | 30.0 | 30.0 |
| | stevioside | 0.5 | 0.5 | 0.1 | 0.1 |
| Viscosity | Agar | 0.3 | — | — | 0.3 |
| controlling | Hydroxyethylene | — | 1.0 | 0.8 | — |
| agent | cellulose | | | | |
| | Povidone K-30 | — | 0.5 | 0.5 | 0.5 |
| | D-sorbitol | 21.0 | 21.0 | 30.0 | 21.0 |
| | solution | | | | |

### TEST EXAMPLE 5

### The Effects of Components of a Dexibupropen Syrup Composition on the Stability and Fluidity

The viscosity, susceptibility to layer separation and fluidity of each of the dexibupropen syrup compositions prepared in Examples 10 to 21 were measured and compared. The results are shown in Table 10.

TABLE 10

| | Ex. | Ex. | Ex. | Ex. | Ex. | Ex. | Ex. | Ex. | Ex. | Ex. | Ex. | Ex. |
|---|---|---|---|---|---|---|---|---|---|---|---|---|
| | 10 | 11 | 12 | 13 | 14 | 15 | 16 | 17 | 18 | 19 | 20 | 21 |
| pH | 3.7 | 3.8 | 3.7 | 3.9 | 3.8 | 3.7 | 3.6 | 3.8 | 3.9 | 3.7 | 3.8 | 3.7 |
| Viscosity | 1,980 | 2,000 | 1,880 | 1,780 | 1,910 | 1,850 | 1,830 | 1,790 | 1,760 | 2,010 | 1,970 | 1,890 |
| (cps) | | | | | | | | | | | | |
| Layer | X | X | X | X | X | X | X | X | X | X | X | X |
| Separation | | | | | | | | | | | | |
| fluidity | Good | Good | Good | Good | Good | Good | Good | Good | Good | Good | Good | Good |

As can be seen in Table 10, excellent syrup compositions showing good fluidity and stability against layer separation and a suitable viscosity can be prepared in accordance with the present invention.

While the invention has been described with respect to the above specific embodiments, it should be recognized that various modifications and changes may be made and also fall within the scope of the invention as defined by the claims that follow.

What is claimed is:

1. A glycerin-free dexibupropen syrup composition comprising dexibupropen ((S)-ibupropen) having an average particle size ranging from 10 to 300 μm as an active ingredient, said composition having a viscosity ranging from 500 to 3,000 cps and pH ranging from 3.0 to 6.0.

2. The composition of claim 1, which further comprises a viscosity controlling agent, a sweetener, a suspending agent, an emulsifier, a pH controlling agent and a preservative.

3. The composition of claim 2, wherein the amounts of the active ingredient, viscosity controlling agent, sweetener, suspending agent, emulsifier and pH controlling agent are 0.1 to 10 w/v %, 0.01 to 40.0 w/v %, 0.1 to 80.0 w/v %, 0.01 to 10.0 w/v %, 0.01 to 5.0 w/v % and 0.01 to 5.0 w/v %, respectively, based on the total volume of the composition.

4. The composition of claim 2, wherein the viscosity controlling agent is selected from the group consisting of D-sorbitol solution, agar, sodium alginate, povidone, polyethylene glycol, hydroxyethylene cellulose and a mixture thereof.

5. The composition of claim 2, wherein the sweetener is selected from the group consisting of a sugar, high fructose, stevioside, dipotassium glycirhyzinate and a mixture thereof.

6. The composition of claim 2, wherein the suspending agent is selected from the group consisting of caoline, xanthan gum, agar and a mixture thereof.

7. The composition of claim 2, wherein the emulsifier is selected from the group consisting of polysorbate compounds.

8. The composition of claim 2, wherein the pH controlling agent is selected from the group consisting of citric acid, sodium citrate and a mixture thereof.

9. The composition of claim 2, which further comprises a preservative selected from the group consisting of methyl parahydroxybenzoate, propyl parahydroxybenzoate, sodium benzoate and a mixture thereof; a flavoring agent; a colorant; or a solvent.

10. A method for the preparation of the composition of claim 1, which comprises the steps of:
(a) dispersing a viscosity controlling agent and a portion of a predetermined amount of a sweetener in distilled water, stirring the resulting mixture at 80 to 90° C. for 1 to 6 hours to obtain a homogeneous solution, adding thereto a preservative, and stirring the resulting solution;
(b) dissolving clearly the remaining portion of the predetermined amount of the sweetener in the solution obtained in (a) while maintaining the solution at 75 to 85° C., adding thereto a viscosity controlling agent and a pH controlling agent, and dissolving the resulting solution completely;
(c) cooling the solution obtained in (b) to 25 to 29° C. by adding cool water;
(d) adding a dexibupropen dispersion obtained by dispersing dexibupropen in a suspending agent to an aqueous emulsion containing a colorant and an emulsifier, and stirring the resulting mixture for 0.5 to 6 hours to obtain a suspension; and
(e) dispersing the solution obtained in (c) in the suspension obtained in (d), adding a flavoring agent, and mixing the resulting mixture uniformly.
